(19) United States
(12) Patent Application Publication
(10) Pub. No.: US 2015/0190208 A1
(43) Pub. Date: Jul. 9, 2015

(54) SYSTEM AND METHOD FOR USER INTERACTION WITH MEDICAL EQUIPMENT

(71) Applicant: Covidien LP, Mansfield, MA (US)

(72) Inventor: Paulo E.X. Silveira, Boulder, CO (US)

(21) Appl. No.: 14/553,101

(22) Filed: Nov. 25, 2014

Related U.S. Application Data
(60) Provisional application No. 61/924,014, filed on Jan. 6, 2014.

(51) Int. Cl. A61B 19/00; A61B 5/117; G06Q 50/22; A61B 5/1455; G06F 19/00; G06Q 30/04; A61B 5/00; A61B 5/0205

(52) U.S. Cl. CPC A61B 19/56; A61B 5/748; A61B 5/117; A61B 5/0205; A61B 5/7282; A61B 5/7425; A61B 5/746; A61B 5/14551; A61B 5/7278; A61B 5/4836; A61B 5/0022; G06F 19/328; G06Q 30/04; G06Q 50/22; A61B 2019/568; A61B 5/02416

(57) ABSTRACT

According to various embodiments, a system may include a user interface configured to detect a first gesture that identifies a specific user. The system may also include a patient monitoring system coupled to user interface that is configured to monitor at least one physiological parameter of a patient. The patient monitoring system may be configured to retrieve and to display one or more event markers for the specific user in response to the detected first gesture. The one or more event markers represent steps of a procedure performed on the patient, and each event marker of the one or more event markers is associated with a respective reimbursement code. The system may convey user identification information associated with the specific user and selected event markers for automatic billing and reimbursement for approved medical procedures.

### CROSS-REFERENCE TO RELATED APPLICATION

This application claims priority to and the benefit of Provisional Application No. 61/924,014, entitled “SYSTEM AND METHOD FOR USER INTERACTION WITH MEDICAL EQUIPMENT”, filed Jan. 6, 2014, which is herein incorporated by reference in its entirety.

### BACKGROUND

The present disclosure relates generally to medical equipment and, more particularly, to user interfaces that enable the identification and/or selection of information associated with the use of the medical equipment.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

An end-user (e.g., clinician or health care provider) utilizing medical equipment (e.g., administration of a particular protocol) may define event markers. These event markers indicate important interventions or steps during a medical procedure. For example, a cardiac procedure (e.g., bypass) may include a variety of steps such as endotracheal intubation, inducing hypothermia, clamping arteries, replacing valves, and/or recovery. Also, these event markers may be linked to reimbursement codes that enable health care providers to charge for specific interventions or events. As a result, making event markers readily available to a health care provider may have important economic consequences. However, different users and different procedures require different event markers. Typically, a large number of event markers are created making it difficult for the user to locate and select the correct marker during a procedure. Often times, the desired event marker may be buried among multiple markers, making selection of a marker a frustrating experience for the user, and, in some cases, preventing the use of the event markers, which may diminish the economic value of utilizing event markers in seeking reimbursement, thus reducing the value of the medical monitoring system.

### BRIEF DESCRIPTION OF THE DRAWINGS

Advantages of the disclosed techniques may become apparent upon reading the following detailed description and upon reference to the drawings in which:

FIG. 1 is a block diagram of a system configured to enable user interaction with a patient monitoring system;

FIG. 2 is a process flow diagram of an embodiment of a method for customizing user interaction with the system of FIG. 1;

FIG. 3 is a process flow diagram of an embodiment of a method for using the system of FIG. 1;

FIG. 4 is diagrammatical view of an embodiment of a user interface (e.g., touchscreen);

FIG. 5 is a diagrammatical view of an embodiment of a user interface (e.g., touch-free gesture recognition user interface);

FIG. 6 is a representation of an embodiment of a screen of a monitor and/or user interface; and

FIG. 7 is a block diagram of an embodiment of a medical device or monitor that may be included in the patient monitoring system of FIG. 1.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Also, as used herein, the term “over” or “above” refers to a component location on a sensor that is closer to patient tissue when the sensor is applied to the patient.

The present embodiments relate to a system that facilitates user interaction with a patient monitoring system and associated medical devices. For example, the system may include a user interface (e.g., touchscreen or touch-free gesture recognition user interface) that facilitates the identification of a specific user, identification and/or selection of specific procedures (e.g., coronary bypass artery surgery or graft or any other procedure), and/or the identification and/or selection of specific event markers (e.g., potential steps to be performed during a procedure) associated with a specific procedure and/or user. The user interface may be coupled to one or more medical devices of the patient monitoring system or integrated within one or more of the medical devices. The user interface may recognize or detect specific gestures that enable the identification and/or selection of the user, procedure, and other information. Once identified, the patient monitoring system may retrieve and display event markers and/or settings (e.g., patient monitoring or medical device settings such as display settings, alarm thresholds, etc.) for the identified user. Also, the patient monitoring system may retrieve and display event markers and/or settings for an identified or selected procedure. The patient monitoring system may associate into a single file values for one or more physiological parameters obtained from the patient undergoing the procedure, user identification information (e.g., user code or ID) associated with the specific user, a procedure code identifying the selected procedure performed on the patient, and/or any event markers selected by the specific user during the procedure. The procedure code and/or event markers may be linked to reimbursement codes that enable health care providers to charge for specific interventions. This system may make it easier and more intuitive for users to identify themselves and to personalize settings when using medical equipment. In addition, the ability to customize with the system facilitates reimbursement by encouraging the use of the event markers.

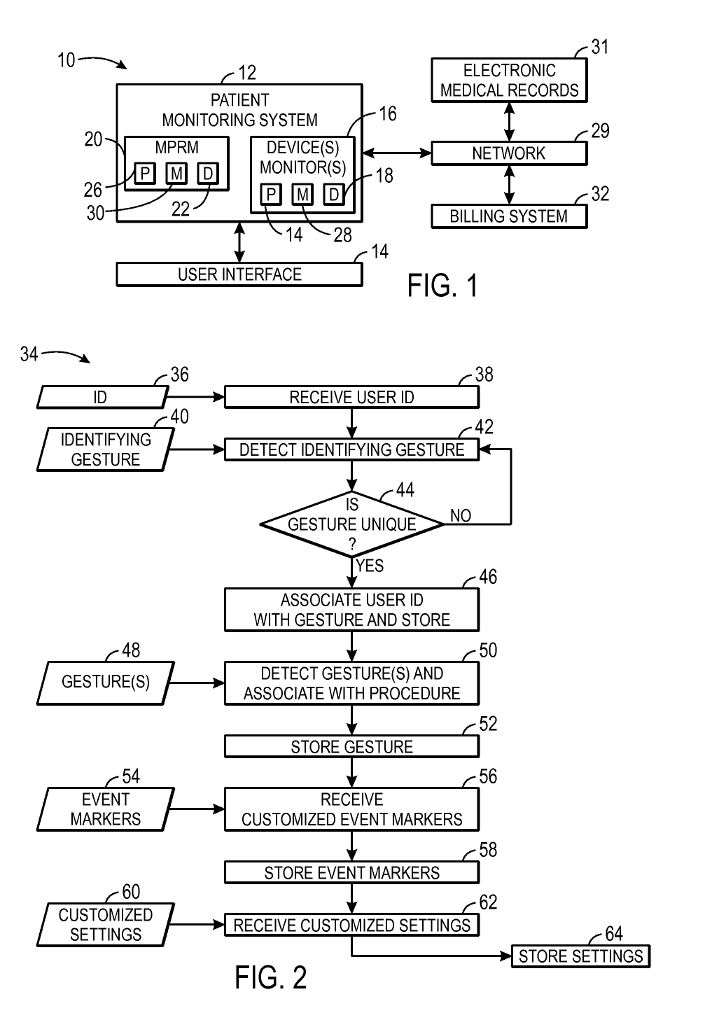

With this in mind, FIG. 1 depicts an embodiment of a system 10 that facilitates user interaction with a patient monitoring system 12 via a user interface 14. The user interface 14 may detect gestures that identify a specific user (e.g., clinician or health care provider). In addition, the detected gestures may be used to identify or select a procedure (e.g., coronary bypass artery surgery or graft or any other procedure) to be performed or being performed on a patient. Further, the detected gestures may be used to identify or select event markers (e.g., potential steps to be performed during a procedure) specific to a procedure and/or user. The user interface 14 may include a touchscreen, touch-free gesture recognition user interface, or a combination thereof. In embodiments where the user interface 14 includes a touchscreen, a detected gesture (e.g., touch or contact based gesture) may include a sequence of touches created by dragging one or more fingers on the touchscreen. In embodiments where the user interface 14 includes a touch-free gesture recognition user interface, the interface 14 may recognize a position and/or movement of one or more fingers and/or the hand.

The patient monitoring system 12 may include one or more medical devices or monitors 16 (e.g., pulse oximeter, regional oximeter, blood pressure device, ventilator, etc.) that may each be configured to monitor one or more physiological parameters (e.g., oxygen saturation, regional oxygen saturation, pulse rate, blood pressure, etc.). The user interface 14 interface may be separate from and coupled to the patient monitoring system 12 (e.g., one or more of the medical devices or monitors 16). In certain embodiments, the patient monitor 16 may be part of or integral to one or more of the medical devices or monitors 16. In embodiments, where the user interface 14 includes a touchscreen, the touchscreen may be part of a respective display 18 of the one or more devices or monitors 16.

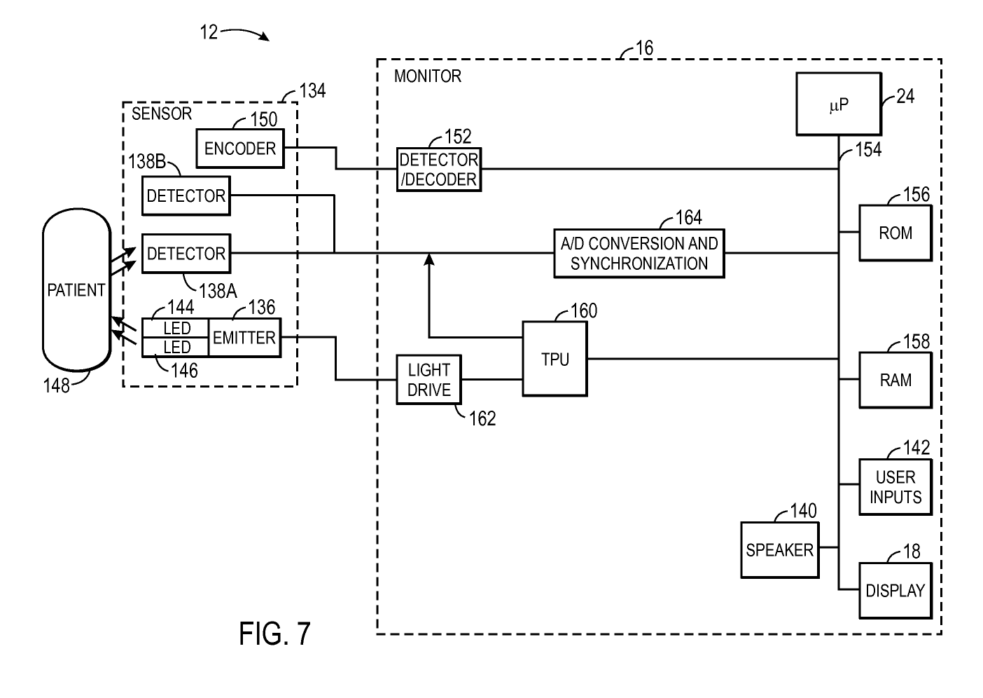

One or more of these devices or monitors 16 may be coupled to one or more sensors (e.g., via a wired or wireless connection) (see FIG. 7). The sensors may generate one or more signals and the monitors 16 may calculate and display (e.g., via a respective display 18) one or more physiological parameters from one or more signals received from the sensors, information about the system, and/or alarm indications. In certain embodiments, one or more of the devices or monitors 16 may be coupled to a multi-parameter patient monitor 20 (e.g., via a wired or wireless connection). The multi-parameter patient monitor 20 may be configured to calculate one or more physiological parameters and to provide a central display 22 for the visualization of information from one or more of the devices or monitors 16. The monitors or devices 16 and/or multi-parameter patient monitor 20 may include various input components knobs, switches, keys and keypads, buttons, touchscreen, scanner, etc., to provide for operation and configuration of the monitors 16, 20. Each device 16 and/or multi-parameter monitor 20 may include one or more respective processors 24, 26 configured to execute code stored on respective memories 28, 30 to calculate the one or more physiological parameters. The processors 24, 26 may also execute code that may utilize the detected gestures to identify a specific user, to select or identify specific procedures, and/or to select specific event markers (i.e., potential steps of a procedure to be performed on a patient). Further, the processors 24, 26 may retrieve and cause to be displayed (e.g., on displays 16, 18) event markers and/or settings (e.g., patient monitoring or medical device settings such as display settings, alarm thresholds, etc.) for the identified user and/or for an identified or selected procedure. The processors 24, 26 may associate into a single file values for one or more physiological parameters obtained from the patient undergoing the procedure, user identification information (e.g., user code or ID) associated with the specific user, a procedure code identifying the selected procedure performed on the patient, and/or any event markers selected by the specific user during the procedure. This file may be stored on the respective memories 28 of the devices or monitors 16 or transferred (e.g., via a wired or wireless connection) to the multi-parameter patient monitor 20. Also, the file may be stored on a removable storage medium (e.g., flash memory, USB flash drive, etc.). In certain embodiments, the devices or monitors 16 and/or the multi-parameter patient monitor 20 may be connected to other systems via a network 29. The devices or monitors 16 and/or multi-parameter patient monitor 20 may be coupled to the network 29 via a physical (e.g., wired or cabled) connection or via a wireless communication technology, such as Wi-Fi, WiMax, Bluetooth, or the like. The network 29 may include one or more servers, which may be configured to facilitate the exchange of information between devices or monitors 16 and/or multi-parameter patient monitor 20. For example, the patient monitoring system 12 may be coupled to an electronic medical records database 31 and/or a billing system 32 via the network 29. The patient monitoring system 12 may transfer the files (including values for one or more physiological parameters obtained from the patient undergoing the procedure, user identification information associated with the specific user, a procedure code identifying the selected procedure performed on the patient, and/or any event markers selected by the specific user during the procedure) to the electronic medical records 31 for storage and/or the billing system 32 to determine reimbursement for the healthcare provider (e.g., via automatic billing for approved medical procedures). The procedure code and/or event markers within each file may be linked to reimbursement codes that enable health care providers to charge for specific interventions.

As discussed above, the system 10 facilitates user interaction with the patient monitoring system 12 via the user interface 14 to make it easier and more intuitive for users to identify themselves and to personalize settings when using medical equipment (e.g., devices or monitors 16). FIG. 2 illustrates a method 34 for how a user interacts with the system 10 to customize it for their use during a medical procedure. The method 34 may begin with the system 10 (e.g., devices or monitors 16) receiving a user ID 36 (e.g., user code and/or other user identification information) (block 38). The user ID 36 may be inputted via input components located on the devices or monitors 16 and/or the user interface 14. In certain embodiments, the user interface 14 may be part of (e.g., makeup part of the user inputs) one or more of the devices or monitors 16. In certain embodiments, the user ID 36 may be entered, for example, using a barcode reader or an RFID tag. After receiving the user ID 36, the user interface 14 may detect an identifying gesture 40 of the user (block 42). For example, the user interface 14 may include a touchscreen that detects various touches or sequence of touches by the user that represent the identifying gesture 40. In some embodiments, the user interface 14 may include a touch-free gesture recognition interface that can recognize a position and/or movement of one or more fingers and/or the hand (i.e., gesture). Upon detecting the identifying gesture 40, the patient monitoring system 12 (e.g., processors 24, 26) may determine whether the identifying gesture 40 was unique relative to other gestures stored on the system 12 (block 44). If the gesture 40 was not unique, the patient monitoring system 12 (e.g., interface 14 and/or monitors 16, 20) may indicate to the user that the gesture 40 was not unique and/or to provide a different identifying gesture 40 for determining its uniqueness. If the gesture 40 is unique, the patient monitoring system 12 may associate or link the gesture 40 with the user ID 36 of the user and store both the user ID and the identifying gesture 40 (e.g., on memories 28, 30).

After associating the identifying gesture 40 with the user ID 36 for the user, the patient monitoring system 12 may receive further gestures 48 from the user via the user interface 14 that may be associated or linked with specific procedures (e.g., procedure codes) (block 50). In certain embodiments, the further gestures 48 may be associated or linked with specific event markers. The gestures 48 (and associated procedure codes) may be stored (e.g., on memories 28, 30) by the patient monitoring system 12 in association with the specific user (block 52). The patient monitoring system 12 may further receive specific customized event markers 54 from the user to be associated with the specific user and/or specific procedure (block 56). The event markers 54 may be inputted by the user via the input components of the monitors 18, 20 and/or selected from a list of potential event markers 54 provided to the user by the monitors 18, 20. The customized event markers 56 may then be stored (e.g., on memories 28, 30) in association with the specific user and/or specific procedure (block 58). The patient monitoring system 12 may further receive customized settings 60 (i.e., user preferred settings) from the user (block 62). The settings 60 may include how different values of one or more of the physiological parameters are displayed (e.g., on displays 18, 22), an order of display for the physiological parameters, one or more alarm thresholds for one or more of the physiological parameters, what indices and ratios to calculate and display, and other settings. The user may provide different settings for different procedures. The customized settings 60 may then be stored (e.g., on memories 28, 30) in association with the specific user and/or specific procedure (block 64).

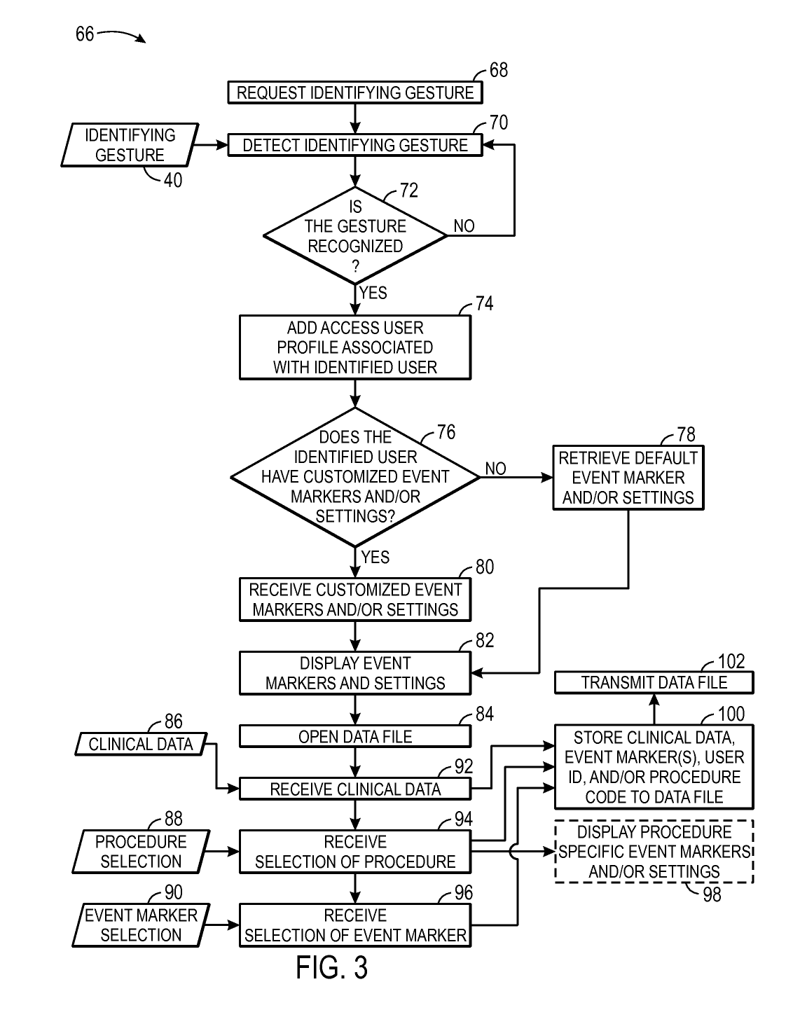

FIG. 3 illustrates a method 66 for how a user interacts with the system 10 during a medical procedure. The method 66 may begin with the patient monitoring system 12 (e.g., monitors 16, 20 or the user interface 14) requesting the identifying gesture 40 for the user (block 68). The user interface 14 may detect the identifying gesture 40 provided by the user (block 70). As mentioned above, the user interface 14 may include a touchscreen that detects various touches or sequence of touches (e.g., touch or contact based gesture) by the user that represent the identifying gesture 40 and/or other gestures 48. In some embodiments, the user interface 14 may include a touch-free gesture recognition interface that can recognize a position and/or movement of one or more fingers and/or the hand (i.e., gesture). Upon detecting the identifying gesture 40, the patient monitoring system 12 (e.g., processors 24, 26) may determine whether the identifying gesture 40 is recognized (block 72). If the gesture 40 is not recognized, the patient monitoring system 12 (e.g., interface 14 and/or monitors 16, 20) may indicate to the user that the gesture 40 was not recognized and/or to provide the identifying gesture 40 again for determining. If the gesture 40 is recognized, the patient monitoring system 12 may access the user profiling (including user ID 36) associated or linked with the gesture 40 (e.g., stored on memories 28, 30) (block 74). In addition, the patient monitoring system 12 (e.g., processors 24, 26) may determine if the identified user has any customized event markers 54 and/or settings 60 associated with the user profile (e.g., stored on memories 28, 30) (block 76). If the identified user does not have any customized event markers 54 and/or settings 60, the patient monitoring system 12 may retrieve default event markers and/or settings (e.g., stored on memories 28, 30) (block 78). If the identified user does have customized event markers 54 and/or settings 60, the patient monitoring system 12 may retrieve the event markers 54 and/or settings 60 (block 80). Upon retrieving the event markers and/or settings (default and/or customized), the patient monitoring system 12 may display them on the user interface 14 and/or displays 18, 22 (block 82).

Prior to, subsequent to, and/or concurrent with retrieving the event markers and/or settings, the patient monitoring system 12 may open a data file (block 84) to associate or store together clinical data 86 (e.g., values of one or more physiological parameters gathered or collected from the patient during a procedure), procedure selections 88 (and associated procedure codes), event marker selections 90, and/or user identification information associated with the identified user (e.g., user ID 36). Upon opening the data file, the patient monitoring system 12 may receive clinical data 86 (e.g., oxygen saturation values, regional saturation values, pulse rate, blood pressure, etc.) gathered from the patient (block 92). In addition, patient monitoring system 12 may receive the selection of one or more procedures 88 from the user (block 94). In certain embodiments, the selection of the procedure 88 may be inputted by the user via input components of the monitors 16, 20. In other embodiments, the selection of the procedure 88 may be made via a gesture detected by the user interface 14. For example, a unique gesture may be made by the user that when detected by the user interface 14 results in the identification or selection of a specific procedure. Alternatively, the user may use a generic gesture that enables scrolling through a list of procedures and then use a subsequent generic gesture that enables the selection of a specific procedure from the list. In certain embodiments, the specific event markers for a user may overlap one or more event markers associated with a specific procedure. Further, the patient monitoring system 12 may receive the selection of one or more event markers 90 from the user (block 96). In certain embodiments, the selection of the event marker 90 may be inputted by the user via input components of the monitors 16, 20. In other embodiments, the selection of the event marker 90 may be made via a gesture detected by the user interface 14. For example, the user may use a generic gesture that enables scrolling through a list of event markers (e.g., specific to the user and/or selected procedure) and then use a subsequent generic gesture that enables the selection of a specific procedure from the list. In certain embodiments the user may use a generic gesture that identifies a specific event marker.

In certain embodiments, the method 66 further includes displaying procedure specific event markers and/or settings based on the selected procedure (block 98). This may be in conjunction with display of user specific event markers and/or settings or in lieu of. The method 66 may also include storing the received clinical data, any selected event marker(s), user ID 36, and/or procedure code for a selected procedure to the single data file (block 100). The single data file may be stored or saved on the memory 28 of the respective device or monitor 16. Also, the file may be stored on a removable storage medium (e.g., flash memory, USB flash drive, etc.). The file may also be transferred (e.g., via a wired or wireless connection) to the multi-parameter patient monitor 20 (block 102) for storage on the memory 30. In addition, the file may be transferred to the electronic medical records database 31 and/or the billing system 32, via the network 29, from the monitors 16, 20. Utilization of the system 10 via these methods 34, 66 may make it easier and more intuitive for users to identify themselves and to personalize settings when using medical equipment. In addition, the ability to customize with the system 10 facilitates reimbursement by encouraging the use of the event markers.

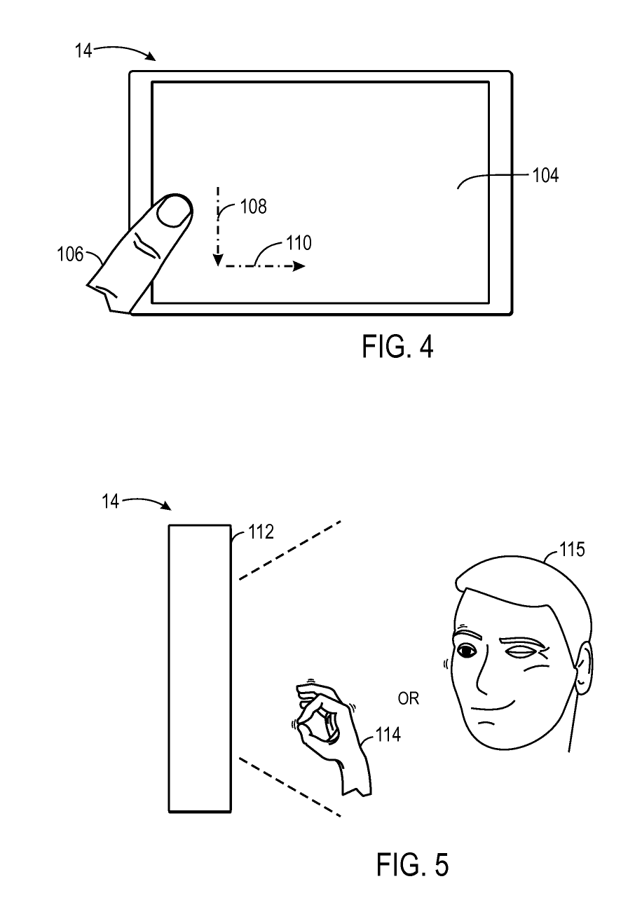

As discussed above, the system 10 includes the user interface 14. FIGS. 4 and 5 illustrate different types of user interfaces 14 and user interaction with them. For example, in FIG. 4 the user interface 14 includes a touchscreen 104 that detects gestures from the user on the touchscreen 104. The gestures may include simple or multi-touch gestures. For example, the touchscreen 104 may detect a gesture from one or more fingers or a stylus 106. The touchscreen 104 may also detect gestures from gloved fingers. The gesture detected by the touchscreen 104 may include one or more movements of any type. The gesture may include movement in one or more directions, including movement of multiple fingers. The gesture, as depicted in FIG. 4, may be as simple as movement in a first direction 108, followed by movement in a second direction 110. The gesture may include any pattern (e.g., circle, square, triangle, X-pattern, etc.). The gesture may also involve a single touch or multiple touches of the touchscreen 104 by the user, using a single or multiple fingers.

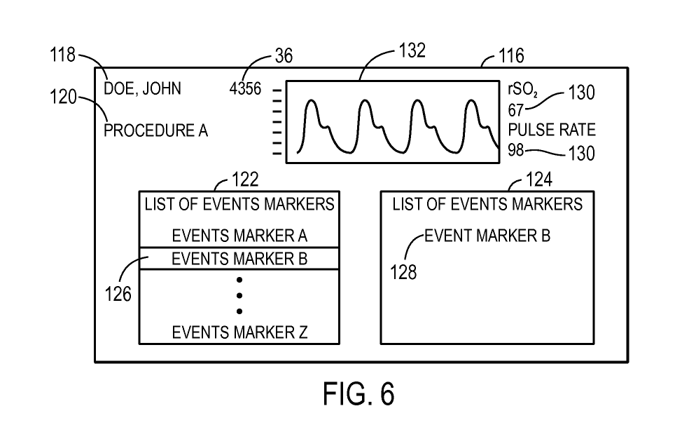

Alternative to or in conjunction with the touchscreen 104, the user interface 14 may include a touch-free gesture recognition user interface 112 as depicted in FIG. 5. The interface 112 may recognize a position and/or movement of one or more fingers and/or the hand 114 of the user without the user touching the interface 112. The interface 112 may also recognize motion or movement of the face 115 (e.g., mouth or lips of the user). The interface 112 may utilize computer vision and/or image processing techniques (e.g., hardware and/or software) to recognize or detect the gesture by the user. Input devices for the interface 112 may include a single standard 2-D camera, stereo cameras, depth-aware cameras, wired gloves, microphones, and/or any other input device that may be used with the interface 112. Various algorithms to interpret the gestures may include 3-D model based algorithms, skeletal-based algorithms, appearance-based algorithms, or any other type of algorithm to interpret the gesture. FIG. 6 illustrates an example of a screen 116 that may be displayed on the user interface 14 and/or the displays 18, 22 of the monitors 16, 20. It should be noted be noted that some or all of the depicted features on the screen 116 may be shown in other embodiments. Other embodiments of the screen 116 may include additional features not shown (e.g., reimbursement codes, additional physiological parameters, thresholds, alarms, trend data, etc.). The name of the user 118 (e.g., clinician, health care provider, etc.) may be shown as well as the user ID 36 on the screen 116. In certain embodiments, information (e.g., name, patient ID, etc.) that identifies a patient undergoing the procedure may be shown. A procedure 120 selected by the user may also be shown. In addition, a list of event markers 122 may be shown on the screen 116. The list of event markers 122 may be specific to the user and/or procedure 120. In addition, a list of selected event markers may be shown on the screen 116. As illustrated, the user may scroll through the list of event markers 122, as described above, to select a desired event marker. For example, the event marker to be selected (e.g., Event Marker B) may highlighted as indicated by numeral 126 and appear among the list of selected event markers 124 as indicated by numeral 128. The list of selected event markers 124 may include one or more selected event markers.

In addition, the screen 116 may also display one or more values 130 for one or more physiological parameters (e.g., regional oxygen saturation, oxygen saturation, pulse rate, blood pressure, hydration level, etc.). In addition, graphs and/or waveforms 132 related to the physiological parameters may be shown on the screen 116. In certain embodiments, other information related to the physiological parameters may be shown. For example, trend data, alarm thresholds, alarms, and other information may be displayed on the screen 116. The physiological parameters and related information may be shown according to the specific settings for the user and/or the procedure.

As described above, one or more medical device or monitors 16 may used in the patient monitoring system 12. FIG. 7 depicts an embodiment of a medical monitor 16 that may be used in conjunction with a medical sensor 134. Although the depicted embodiments relate to sensors for use on a patient's head, it should be understood that, in certain embodiments, the features of the sensor 134 as provided herein may be incorporated into sensors for use on other tissue locations, such as the back, the stomach, the heel, the ear, an arm, a leg, or any other appropriate measurement site. In addition, although the embodiment of the patient monitoring system 12 illustrated in FIG. 7 relates to photoplethysmography or regional oximetry, the system 12 may be configured to obtain a variety of medical measurements with a suitable medical sensor. For example, the system 12 may additionally be configured to determine patient electroencephalography (e.g., a bispectral index), or any other desired physiological parameter such as water fraction, end-title CO₂, or hematocrit.

As noted, the system 12 includes the sensor 134 that is communicatively coupled to the patient monitor 16. The sensor 134 may be reusable, entirely disposable, or include disposable portions. If the sensor 134 is reusable, it may include a disposable adhesive pad that may be replaced. Although only one sensor 134 is shown coupled to the monitor 16 in FIG. 7, in other embodiments, two, three, four, or more sensors 134 may be coupled to the monitor 16. For example, two sensors 134 may be used for cerebral oximetry and simultaneously two other sensors 134 used for somatic oximetry. As shown in FIG. 7, the sensor 134 includes an emitter 136 and a pair of detectors 138 (e.g., 138A, 138B). The emitter 136 and detectors 138 of the sensor 134 may be coupled to the monitor 16 via a cable. The cable may interface directly with the sensor 134 and may include a plurality of conductors (e.g., wires). In certain embodiments, the sensor 134 may be configured to store patient-related data, such as historical regional oximetry data (e.g., rSO₂ values).

The monitor 16 may be any suitable monitor, such as an INVOS® System monitor available from Covidien Corporation. The monitor 16 includes the monitor display 18 configured to display information regarding the physiological parameters monitored by the sensor 134, information about the system, and/or alarm indications. In addition, the monitor 134 may display information related to the user, a selected procedure, potential event markers, selected event markers, and other information (see FIG. 6). The monitor 16 may also include a speaker 140 to communicate information related to the physiological parameters (e.g., alarms). The monitor 16 may include various input components 142, such as knobs, switches, keys and keypads, buttons, touchscreen, etc., to provide for operation and configuration of the monitor 16. The input components 142 may enable the inputting of user information and/or selection of procedures and/or event markers. In certain embodiments, the display 18 may be used as the user interface 14 described above. The monitor 16 also includes the processor 24 that may be used to execute code, such as code for implementing various monitoring functionalities enabled by the sensor 134. As discussed below, for example, the monitor 16 may be configured to process signals generated by the detectors 134 to estimate the amount of oxygenated vs. de-oxygenated hemoglobin in a monitored region of the patient (e.g., brain). In some embodiments, the sensor 134 may include a processor that may be used to execute code stored in a memory of the sensor 134 to perform all or some of the functionalities described throughout related to calculating an rSO₂ value.

The monitor 16 may be any suitable monitor, such as an INVOS® System monitor available from Covidien Corporation. In certain embodiments, the sensor 134 may be a wireless sensor 134. Accordingly, the wireless sensor 134 may establish a wireless communication with the patient monitor 16, the multi-parameter patient monitor 20, and/or network 29 using any suitable wireless standard. In certain embodiments, a pre-amplifier may be utilized between the sensor 134 and monitor 16. In this embodiment, wireless communication may occur between the pre-amplifier, sensor 134, monitor 20, and/or the network 29. By way of example, the wireless module may be capable of communicating using one or more of the ZigBee standard, WirelessHART standard, Bluetooth standard, IEEE 802.11x standards, or MiWi standard.

As provided herein, the sensor 134 may be configured to perform regional oximetry. Indeed, in one embodiment, the sensor 134 may be an INVOS® cerebral/somatic sensor available from Covidien Corporation. In regional oximetry, by comparing the relative intensities of light received at two or more detectors, it is possible to estimate the blood oxygen saturation of hemoglobin in a region of a body. For example, a regional oximeter may include a sensor to be placed on a patient's forehead and may be used to calculate the oxygen saturation of a patient's blood within the venous, arterial, and capillary systems of a region underlying the patient's forehead (e.g., in the cerebral cortex). As illustrated in FIG. 7, the sensor 134 may include the emitter 136 and the two or more detectors 138: one detector 138A that is relatively “close” to the emitter 136 and another detector 138B that is relatively “far” from the emitter 136. Light intensity of one or more wavelengths may be received at both the “close” and the “far” detectors 138A and 138B. Thus, the detector 138A may receive a first portion of light and the detector 138B may receive a second portion of light. Each of the detectors 138 may generate signals indicative of their respective portions of light. For example, the resulting signals may be contrasted to arrive at a regional saturation value that pertains to additional tissue through which the light received at the “far” detector 138B passed (tissue in addition to the tissue through which the light received by the “close” detector 138A passed, e.g., the brain tissue) when it was transmitted through a region of a patient (e.g., a patient's cranium). Surface data from the skin and skull is subtracted out to produce a regional oxygen saturation (rSO₂) value for deeper tissues.

The emitter 136 and the detectors 138 may be arranged in a reflectance or transmission-type configuration with respect to one another. However, in embodiments in which the sensor 134 is configured for use on a patient's forehead, the emitter 136 and detectors 138 may be in a reflectance configuration. An emitter 136 may also be a light emitting diode, superluminescent light emitting diode, a laser diode, or a vertical cavity surface emitting laser (VCSEL). An emitter 136 and the detectors 138 may also include optical fiber sensing elements. Also, the emitter 136 may include two light emitting diodes (LEDs) 144 and 146 that are capable of emitting at least two wavelengths of light, e.g., red or near infrared light. In one embodiment, the LEDs 144 and 146 emit light in the range of about 600 nm to about 1000 nm. In a particular embodiment, the one LED 144 is capable of emitting light at 730 nm and the other LED 146 is capable of emitting light at 810 nm. In another particular embodiment, the emitter 136 may include four LEDs configured to emit at least four wavelengths of light of peak wavelengths of approximately 730 nm, 770 nm, 810 nm and 850 nm. It should be understood that, as used herein, the term “light” may refer to one or more of ultrasound, radio, microwave, millimeter wave, infrared, near-infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In any suitable configuration of the sensor 134, the detectors 138A and 138B may be an array of detector elements that may be capable of detecting light at various intensities and wavelengths. In one embodiment, light enters the detector 138 (e.g., detector 138A or 138B) after passing through the tissue of the patient 148. In another embodiment, light emitted from the emitter 136 may be reflected by elements in the patient's tissue to enter the detector 138. The detector 138 may convert the received light at a given intensity, which may be directly related to the absorbance and/or reflectance of light in the tissue of the patient 148, into an electrical signal. That is, when more light at a certain wavelength is absorbed, less light of that wavelength is typically received from the tissue by the detector 138, and when more light at a certain wavelength is reflected, more light of that wavelength is typically received from the tissue by the detector 138. After converting the received light to an electrical signal, the detector 138 may send the signal to the monitor 16, where physiological characteristics may be calculated based at least in part on the absorption and/or reflection of light by the tissue of the patient 148.

In certain embodiments, the medical sensor 134 may also include an encoder 150 that may provide signals indicative of the wavelength of one or more light sources of the emitter 136, which may allow for selection of appropriate calibration coefficients for calculating a physical parameter such as blood oxygen saturation. The encoder 150 may, for instance, include a coded resistor, an electrically erasable programmable read only memory (EEPROM), or other coding device (such as a capacitor, inductor, programmable read only memory (PROM), RFID, parallel resident currents, or a colorimetric indicator) that may provide a signal to the microprocessor 24 related to the characteristics of the medical sensor 134 to enable the microprocessor 24 to determine the appropriate calibration characteristics of the medical sensor 134. Further, the encoder 150 may include encryption coding that prevents a disposable part of the medical sensor 134 from being recognized by a microprocessor 24 unable to decode the encryption. For example, a detector/decoder 152 may translate information from the encoder 150 before the processor 24 can properly handle it. In some embodiments, the encoder 150 and/or the detector/decoder 152 may not be present.

In certain embodiments, the sensor 134 may include circuitry that stores patient-related data (e.g., rSO₂) and provides the data when requested. The circuitry may be included in the encoder 150 or in separate memory circuitry within the sensor 134. Examples of memory circuitry include, but are not limited to, a random access memory (RAM), a FLASH memory, a PROM, an EEPROM, a similar programmable and/or erasable memory, any kind of erasable memory, a write once memory, or other memory technologies capable of write operations. In one embodiment, patient-related data, such as the rSO₂ values, trending data, or patient monitoring parameters, may be actively stored in the encoder 150 or memory circuitry.

Returning to FIG. 7, signals from the detector 138 and/or the encoder 150 may be transmitted to the monitor 16. The monitor 16 may include one or more processors 24 coupled to an internal bus 154. Also connected to the bus 154 may be a ROM memory 156, a RAM memory 158, and the display 18. A time processing unit (TPU) 160 may provide timing control signals to light drive circuitry 162, which controls when the emitter 136 is activated, and if multiple light sources are used, the multiplexed timing for the different light sources. The received signal from the detector 138 may be passed through analog-to-digital conversion and synchronization 164 under the control of timing control signals from the TPU 160. Specifically, the signal may undergo synchronized demodulation and optionally amplification and/or filtering. For example, the LEDs 144 and 146 may be driven out-of-phase, sequentially and alternatingly with one another (i.e., only one of the LEDs 144 and 146 being driven during the same time interval) such that the detector 138 receives only resultant light spectra emanating from one LED at a time. Demodulation of the signal enables the data associated with the LEDs 144 and 146 to be distinguished from one another. After demodulation, the digital data may be downloaded to the RAM memory 158.

In some embodiments, the processor 24 may execute code that utilizes the detected gestures to identify a specific user, to select or identify specific procedures, and/or to select specific event markers (i.e., potential steps of a procedure to be performed on a patient). Further, the processor 24 may retrieve and cause to be displayed (e.g., on display 18) event markers and/or settings (e.g., patient monitoring or medical device settings such as display settings, alarm thresholds, etc.) for the identified user and/or for an identified or selected procedure. The processor 24 may associate into a single file values for one or more physiological parameters obtained from the patient undergoing the procedure, user identification information (e.g., user code or ID) associated with the specific user, a procedure code identifying the selected procedure performed on the patient, and/or any event markers selected by the specific user during the procedure.

In an embodiment, based at least in part upon the received signals corresponding to the light received by detector 138, the processor 24 may calculate the oxygen saturation (e.g., regional oxygen saturation) using various algorithms. These algorithms may use coefficients, which may be empirically determined. For example, algorithms relating to the distance between an emitter 136 and various detector elements in a detector 138 may be stored in the ROM memory 156 and accessed and operated according to processor instructions. Additionally, algorithms may use the value of LED wavelengths encoded in sensor encoder 150, enabling the algorithm to compensate for LED wavelengths that diverge from nominal wavelengths.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A system comprising:
a user interface configured to detect a first gesture that identifies a specific user; and
a patient monitoring system coupled to the user interface and configured to monitor at least one physiological parameter of a patient;
wherein the patient monitoring system is configured to retrieve and to display one or more event markers for the specific user in response to the detected identifying gesture, the one or more event markers representing steps of a procedure performed on the patient, each event marker of the one or more event markers is associated with a respective reimbursement code;
wherein the patient monitoring system is configured to associate user identification information associated with the specific user with any event marker selected by the specific user during the procedure; and
a billing system configured to receive the user identification information associated with the specific user and any event marker selected by the specific user during the procedure, and the billing system is configured to generate a bill utilizing one or more reimbursement codes associated with one or more respective selected event markers.

2. The system of claim 1, wherein the user interface is configured to detect a second gesture, and wherein the second gesture identifies a corresponding procedure to be performed on the patient.

3. The system of claim 2, wherein the patient monitoring system is configured to retrieve and to display specific event markers associated with the corresponding procedure.

4. The system of claim 1, wherein the patient monitoring system is configured to determine if the first gesture is unique relative to other gestures and to associate the first gesture with the specific user if the first gesture is unique.

5. The system of claim 1, wherein the patient monitoring system is configured to retrieve and to display patient monitoring system settings customized for the specific user in response to the first gesture.

6. The system of claim 5, wherein the patient monitoring systems settings comprises at least one of how different values of the at least one physiological parameter are displayed, one or more alarm thresholds for the at least one physiological parameter, an order of display for a plurality of physiological parameters, which physiological parameters of the plurality of physiological parameters to display, or a combination thereof.

7. The system of claim 1, wherein the patient monitoring system comprises at least one medical device to monitor the at least one physiological parameter, and the at least one medical device comprises the user interface.

8. The system of claim 7, wherein the at least one medical device comprises a pulse oximetry monitor or a regional oximetry monitor.

9. The system of claim 1, wherein the first gesture comprises a touch-based gesture and user interface comprises a touchscreen configured to detect the touch-based gesture.

10. A monitor comprising:
a user interface configured to detect a first gesture that identifies a specific user; and
a processing device coupled to the user interface and configured to retrieve one or more event markers for the specific user in response to the detected first gesture, the one or more event markers representing steps of a procedure performed on a patient, and the processing device is configured to associate user identification information associated with the specific user with any event markers selected by the specific user during the procedure.

11. The monitor of claim 10, comprising a port configured to couple to a sensor applied to the patient, wherein the processing device is configured to receive a signal from the sensor and to calculate at least one physiological parameter from the received signal, and the processing device is configured to associate values of the at least one physiological parameter obtained from the patient with the user identification information and selected event markers.

12. The monitor of claim 10, wherein the user interface is configured to detect a second gesture, and wherein the second gesture identifies a corresponding procedure to be performed on the patient and causes the processing device to retrieve a code for the corresponding procedure identified.

13. The monitor of claim 12, wherein the processing device is configured to retrieve specific event markers associated with the corresponding procedure identified.

14. The monitor of claim 11, wherein first gesture comprises a touch-based gesture, and the user interface comprises a touchscreen configured to detect the touch-based gesture.

15. The monitor of claim 10, wherein the processing device is configured to determine if the first gesture is unique relative to other gestures and to associate the first gesture with the specific user if the first gesture is unique.

16. A method comprising:
detecting, via a user interface, identifying first gesture that identifies a specific user;
retrieving and displaying, via a patient monitor, one or event markers for the specific user in response to the detected first gesture, wherein the one or more event markers represent steps of a procedure performed on a patient; and
associating, via the patient monitor, user identification information associated within the specific user with any event makers selected by the specific user during the procedure.

17. The method of claim 16, comprising detecting, via the user interface, a second gesture that identifies and selects a corresponding procedure to be performed on the patient.

18. The method of claim 16, comprising determining, via the patient monitor, if the first gesture is unique relative to other gestures and to associate the first gesture with the specific user if the identifying gesture is unique.

19. The method of claim 16, wherein each event marker of the one or more event markers is associated with a respective reimbursement code.

20. The method of claim 16, comprising receiving the user identification information associated with the specific user and any event marker selected by the specific user during the procedure, and generating a bill utilizing one or more reimbursement codes associated with one or more respective selected event markers.
